(19) United States
(12) Patent Application Publication
(10) Pub. No.: US 2022/0354930 A1
(43) Pub. Date: Nov. 10, 2022

(54) Hemoglobin Substitute Mixtures Including Reconstituted Plasma and Platelets and Their Manufacture and Use

(71) Applicants: HbO2 Therapeutics, LLC, Souderton, PA (US); The Government of the United States, as Represented by the Secretary of the Army, Fort Detrick, MD (US)

(72) Inventors: Gregory P. Dubé, Littleton, MA (US); Michael A. Meledeo, San Antonio, TX (US); Andrew P. Cap, San Antonio, TX (US)

(21) Appl. No.: 17/623,536

(22) PCT Filed: Jul. 2, 2020
(86) PCT No.: PCT/US2020/040698

§ 371 Date: Dec. 28, 2021

Related U.S. Application Data
(60) Provisional application No. 62/869,825, filed on Jul. 2, 2019.

(51) Int. Cl. A61K 38/42; A61K 47/42; A61K 35/16; A61K 35/19; A61K 47/02; A61P 7/08; A61K 9/00

(52) U.S. Cl. CPC A61K 38/42; A61K 47/42; A61K 35/16; A61K 35/19; A61K 47/02; A61P 7/08; A61K 9/0026

(57) ABSTRACT

A therapeutic hemoglobin-based oxygen carrier solution is formed by directly combining at least one of freeze-dried platelets and freeze-drug plasma with a hemoglobin-based oxygen carrier. The therapeutic hemoglobin-based oxygen carrier solution can be employed to treat bleeding or anemia and simultaneously increase systemic convective oxygen delivery in a subject suffering low circulatory oxygen transport or bleeding.

### RELATED APPLICATION(S)

This application claims the benefit of U.S. Provisional Application No. 62/869,825, filed on Jul. 2, 2019; the relevant teachings of which are incorporated by reference in their entirety.

### STATEMENT OF GOVERNMENT INTEREST

This invention was made with government support under Grant W81XWH-15-0269 awarded by the Government of the United States, as Represented by the U.S. Army Medical Research and Development Command, Institute of Surgical Research. The government has certain rights in the invention.

### BACKGROUND OF THE INVENTION

Hemorrhagic trauma can result in morbidity or death unless the patient's anemia and hypovolemia can be rapidly reversed and hemostasis can be achieved and maintained to limit or eliminate further bleeding. Intervention to achieve these objectives typically involve intravenous (IV) infusion of stored red blood cells (oxygen carrier), plasma (containing coagulation factors that form a clot in the wound) and platelets (specialized blood cells that clump together to strengthen the clot). However, transport and storage of these traditional blood products requires refrigeration. In far-forward military conflicts and remote civilian injury scenes, cold-stored blood products are often unavailable because refrigeration capability (“cold chain”) does not exist. Such austere and remote environments often preclude timely evacuation of the injured to higher level care, further elevating their risk of morbidity and death. Both hemoglobin-based oxygen carriers (HBOCs) and freeze-dried plasma (FDP) (until rehydrated in a glass vial with sterile water)¹ are stable for years at room temperature are currently available in the U.S. under an Emergency IND or Emergency Use Authorization and can be transported to combat arenas and remote civilian injury sites by medics in the absence of a cold chain. Simultaneous administration of separate HBOC and rehydrated FDP products requires establishment of two intravenous (IV) access sites in patients who, because of their physiological status, often make IV access difficult.

Use of HBOCs to treat severe anemia during and following hemorrhagic trauma has been accompanied by coagulopathy.² The basis for HBOC-associated coagulopathy likely involves, in part, dilution of residual plasma coagulation factors in circulation.^{3,3} However, thromboelastography (TEG) in vitro experiments implicate additional mechanisms, including increased fibrinolysis via stimulation of a tissue plasminogen activator (tPA)-dependent mechanism as well as via an unidentified, tPA-independent mechanism.⁴ Multiple resuscitation fluids (normal saline, lactated Ringer's, 6% albumin, Plasma-Lyte, Hextend, Voluven and 6% dextran) induce tPA-dependent fibrinolysis, suggesting a common mechanism such as dilution of fibrinolysis inhibitors, alpha-2 antiplasmin⁵ and plasminogen activator inhibitor-1 (PAI-1).^{6, 7, 8, 9, 10} Inappropriately elevated tPA-dependent fibrinolysis can be blocked by administration of tranexamic acid (TXA), a low molecular weight drug that blocks plasminogen activation by tPA, but no known mechanism exists to limit HBOC-induced tPA-independent fibrinolysis.

Although the increase in tPA-independent fibrinolysis by HBOC in an in vitro study⁵ was small (5%) compared to the 44% increase in tPA-dependent fibrinolysis, a 3% increase in fibrinolysis has been shown to substantially increase mortality in severely injured trauma patients.¹¹ Thus, in serious trauma cases, improvements in oxygen transport made possible by HBOC-201 administration may be offset or even overwhelmed by exacerbated blood loss in a patient already struggling with elevated tPA-dependent fibrinolysis.

In addition to their effects on fibrinolysis, HBOCs have been shown to inhibit clot formation as measured by TEG.^{12, 13} Increasing concentrations of cell-free Hb by mixing Oxyglobin, an HBOC closely related to HBOC-201, with whole blood results in an HBOC dose-dependent deterioration in clotting parameters including the alpha angle (a measure of clot propagation rate), MA (maximum amplitude, an indicator of clot size) and G, (an exponential expression of MA) reported by TEG analysis. The impact on clot formation is even greater if the cell-free Hb source added to blood has a high metHb content. Severely anemic patients (characterized by very low hematocrit), those most in need of HBOC infusion when traditional blood products are not available, tend to develop relatively high metHb concentrations due to low levels of cytochrome b5 NADH-dependent metHb reductase contained in red blood cells (RBCs).^{13, 14} NADH-dependent metHb reductase is the enzyme primarily responsible for reducing metHb(Fe³⁺) back to Hb(Fe²⁺) following normal auto-oxidation of Hb in the presence of oxygen.¹⁵

The specific mechanism(s) by which HBOCs inhibit clot formation are unknown. However, reactive oxygen species (ROS), ferryl and ferryl radical, resulting from the reaction of metHb with H₂O₂, may play a role. These ROS, characterized by high redox potentials, are capable of modifying tissues and hemoglobin itself, resulting in the release of heme that may become oxidized to the pro-inflammatory mediator, hematin, in the presence of metHb.^{15, 16} ROS-induced tissue damage, and protein modification could potentially perturb the normal function of one or more coagulation proteins or platelet aggregatory activity.¹⁷ In addition, the cell-free Hb contained in HBOCs reacts rapidly and irreversibly with nitric oxide (NO) to yield metHb which in turn, can further react with NO to form NO—Fe complexes' These reactions are capable of further reducing bioavailable NO and altering the balance of NO-containing redox mediators including glutathione, nitrite, nitrate, and S-nitrosothiols (SNOs), which include S-nitrosylated cysteine residues in serum proteins, and S-nitrosoglutathione (GSNO) in the cytoplasm.^{17, 18}

Platelet aggregation plays an important role in thrombus propagation¹⁹ and HBOC administration is known to inhibit the role of platelet aggregation in vivo.³ Platelets contain Ib cell adhesion receptors and αIIbβ3 integrin receptors that are capable of binding fibrinogen²⁰ and contain vicinal thiol moieties that are critically important in mediating platelet aggregation.²¹ Vicinal thiols are redox-sensitive sites capable of interacting with endogenous reducing agents including SNOs.²² Platelets also contain P2Y12 ADP receptors that mediate platelet activation in response to ADP and other platelet activators. P2Y12 receptors contain extracellular thiols that may be regulatory nitrosylation targets for NO or SNO molecules. Depletion of NO and SNO through cell-free Hb- and ROS-mediated mechanisms previously described may inhibit normal P2Y12 receptor function, thereby inhibiting platelet aggregation.

Thus, the published studies described above concerning in vitro combinations of HBOCs and blood or the administration of HBOCs systemically raise the expectation that reconstituting freeze-dried plasma or freeze-dried platelets with an HBOC solution will inhibit clot formation, enhance clot lysis and exacerbate coagulopathy associated with severe hemorrhagic trauma. For these reasons, administering an HBOC-rehydrated FDP or FDPlt would appear to be contraindicated in an individual at risk for or already compromised by coagulopathy.

US 2007/0265195 A1 describes “multifunctional blood substitutes” that include, for example, a hemoglobin-based oxygen carrier (HBOC), such as HBOC-201 combined with and infusible platelet membrane (IPM) that has been freeze-dried.²² However, the data presented in US 2007/0265195 A1 shows suppression of both platelet function and TEG MA by addition of HBOC to the assay mixture, consistent with the inhibitory effects of HBOCs on platelet function and coagulation reported in the literature reviewed above. The teaching of US 2007/0265195 A1 is deficient by failing to evaluate or provide a method of preserving platelet function and other clot formation processes in the presence of added HBOC. Nor do the teachings of US 2007/0265195 A1 identify any such work or data from others addressing this issue.

Therefore, a need exists to overcome or minimize the above-referenced problems.

### SUMMARY OF THE INVENTION

The invention generally relates to a method of forming a therapeutic hemoglobin-based oxygen carrier solution, the hemoglobin-based solution formed by the method, and to a method of treating bleeding or anemia and simultaneously increasing systemic convective oxygen delivery in a subject suffering from low circulatory oxygen transport or bleeding by use of the therapeutic hemoglobin-based oxygen carrier solution of the invention.

In one embodiment, the invention is directed to a method of forming a therapeutic hemoglobin-based oxygen carrier solution that includes the step of directly combining at least one of freeze-dried platelets and freeze-dried plasma with a hemoglobin-based oxygen carrier to form a therapeutic hemoglobin-based oxygen carrier solution.

In yet another embodiment, the invention is directed to a therapeutic hemoglobin-based oxygen carrier solution formed by a method comprising the step of directly combining at least one of freeze-dried platelets and freeze-dried plasma with a hemoglobin-based oxygen carrier to form a therapeutic hemoglobin-based oxygen carrier solution.

In still another embodiment, the invention is directed to a method of treating bleeding or anemia and simultaneously increasing systemic convective oxygen delivery and in a subject suffering from low circulatory oxygen transport or bleeding, the method including the step of administering to a subject in need thereof a therapeutically effective amount of a therapeutic hemoglobin-based oxygen carrier solution formed by directly combining at least one of freeze-dried platelets and freeze-dried plasma with a hemoglobin-based oxygen carrier.

This invention has many advantages. For example, rehydrating FDP (packaged in an infusion bag rather than glass vials) with HBOC-201 rather than sterile water, in the field at the time of need, yields a combination product capable of resolving anemia, restoring circulatory volume and minimizing or eliminating coagulopathy. The combination product will also significantly reduce the weight and volume of medical materials (sterile water and glass containers) that present logistical challenges to dismounted medics in the field and will be deliverable via one infusion line, a significant advantage over infusing separate products via two lines under patient and operational conditions that complicate establishment of intravenous (IV) access.²³

Reconstitution of FDPlt with HBOC-201 produces an on-demand combination product that can be administered in the field, has the advantages of being stable without refrigeration, having lower weight and volume than fresh or frozen platelets, and of being administered via one intravenous access site, similar to the HBOC-FDP combination product of the invention.

In another embodiment, FDP and FDPlt are both combined, at the time of need, with HBOC-201 in one package (triple combination product). Such a product closely simulates the oxygen-carrying and hemostasis properties of whole blood, providing greater efficacy (decreased morbidity and mortality) in treating hemorrhagic trauma and further reduction in product weight and volume compared to use of the corresponding component products separately.

Addition of HBOC-rehydrated FDP reduces dilution-dependent declines in platelet concentrations and preserves TEG MA compared to addition of HBOC alone or water-rehydrated HBOC. Rehydrating FDP with HBOC, such as, for example, HBOC from which all low molecular weight (<30 kD) components have been reduced except calcium, improves the rate of clot propagation (as indicated by TEG alpha angle) and TEG MA. Thus, the method of the invention and the therapeutic hemoglobin-based oxygen carrier solution of the inventions reduces or eliminates the negative impacts of HBOC on clot formation processes reported in the scientific literature.

### BRIEF DESCRIPTION OF THE DRAWINGS

FIG. 1 is a schematic drawing of field-ready flexible containers to hold a polymerized hemoglobin solution and, separately, freeze-dried plasma, freeze-dried platelets or a combination of freeze dried plasma and freeze-dried platelets.

FIG. 2 is a schematic drawing of a transfer tube with spike ports for making secure leak-proof connections between a flexible container of polymerized hemoglobin solution and a flexible container of freeze-dried plasma, freeze-dried platelets or a combination of freeze-dried plasma and freeze-dried platelets.

FIG. 3 is a schematic drawing of a field-ready flexible container system designed to facilitate transfer of polymerized hemoglobin solution from its flexible container to a flexible container of freeze-dried plasma, freeze-dried platelets or combination freeze-dried plasma and freeze-dried platelets, and subsequent administration of rehydrated therapeutic to a patient.

FIG. 4 is a schematic drawing of an infusion tube equipped with spike end and hypodermic needle to enable administration of solutions to patients of polymerized hemoglobin-rehydrated freeze-dried plasma, freeze-dried platelets or combination freeze dried plasma and freeze-dried platelets.

FIG. 5 is a schematic drawing of a field-ready flexible container system designed to facilitate sequential transfer of polymerized hemoglobin solution from its flexible container to flexible containers of freeze-dried plasma and freeze-dried platelets, and subsequent administration of rehydrated therapeutic to a patient.

FIG. 6 is a schematic drawing of a field-ready flexible container system designed to facilitate sequential transfer of polymerized hemoglobin solution from its flexible container to a flexible container of freeze-dried plasma and freeze-dried platelets, and subsequent administration of rehydrated therapeutic to a patient.

FIG. 7A is a histogram showing the effect of 50% volume replacement of whole blood on hemoglobin concentration when employing freeze-dried plasma rehydrated in water according to the prior art.

FIG. 7B is a histogram showing the effect of 50% volume replacement of whole blood on hemoglobin concentration employing freeze-dried plasma rehydrated in a hemoglobin-based oxygen carrier according to one embodiment of the invention.

FIG. 8A is a histogram showing the effect of 50% whole blood volume replacement on platelet concentration employing freeze-dried plasma rehydrated in water according to the prior art.

FIG. 8B is a histogram showing the effect of 50% whole blood volume replacement on platelet concentration employing freeze-dried plasma rehydrated in a hemoglobin-based oxygen carrier according to an embodiment of the invention.

FIG. 9A is a histogram showing the effect of 50% whole blood volume replacement on fibrinogen concentration employing freeze-dried plasma rehydrated in water according to the prior art.

FIG. 9B is a histogram showing the effect of 50% whole blood volume replacement on fibrinogen concentration employing freeze-dried plasma rehydrated in a hemoglobin-based oxygen carrier according to an embodiment of the invention.

FIG. 10A is a histogram showing the effect of dose-dependent dilution of whole blood by hemoglobin-based oxygen carrier-rehydrated freeze-dried plasma on fibrinogen concentration according to an embodiment of the invention, both with and without employment of a diluted 25% Plasma-Lyte crystalloid intravenous infusion resuscitation fluid (Baxter International, Inc., Deerfield, Ill.).

FIG. 10B is a histogram showing the effect of dose-dependent dilution of whole blood by hemoglobin-based oxygen carrier-rehydrated freeze-dried plasma on prothrombin time (PT) according to an embodiment of the invention, both with and without employing a 25% Plasma-Lyte resuscitation fluid.

FIG. 10C is a histogram showing the effect of dose-dependent dilution of whole blood by hemoglobin-based oxygen carrier-rehydrated freeze-dried plasma on activated partial thromboplastin time (aPTT) according to an embodiment of the invention, both with and without employing 25% Plasma-Lyte resuscitation fluid.

FIG. 11 is a histogram showing the effect of whole blood replacement by hemoglobin-based oxygen carrier-rehydrated freeze-dried plasma according to embodiment of the invention on thromboelastography (TEG) alpha angle.

FIG. 12A is a histogram showing the effect of a 50% volume replacement of whole blood on TEG maximum amplitude employing freeze-dried plasma that has been rehydrated in water according to the prior art.

FIG. 12B is a histogram showing the effect of 50% volume replacement of whole blood on TEG maximum amplitude employing freeze-dried plasma rehydrated in a hemoglobin-based oxygen carrier according to an embodiment of the invention.

FIG. 13 is a histogram showing the effect of 50% whole blood volume replacement by hemoglobin-based oxygen carrier-rehydrated freeze-dried plasma of one embodiment of the invention on TEG percent clot lysis 60 minutes after initiation of the clotting reaction (LY60).

FIGS. 14A-14E are histograms showing the effect of whole blood versus partial whole blood replacement by various formulations of hemoglobin-based oxygen carrier (HBOCs) and freeze-dried plasma, both according to the prior art (water-based, or WB), and by use of various embodiments of the invention, representing in FIG. 14A the R-time in minutes, in FIG. 14B the maximum amplitude in millimeters, in FIG. 14C the angle in degrees, in FIG. 14D percent clot lysis 30 min after initiation of the clotting reaction (LY30) in percent, and in FIG. 14E, the LY60, also in percent, all of which results are presented with and without the presence of the presence of plasminogen activator (tA).

FIGS. 15A-15C are histograms of fibrinogen (FIG. 15A), PT (FIG. 15B), and aPTT (FIG. 15C) of whole blood, versus partial whole blood replacement by various formulations, both prior art (WB) and various embodiments of the invention, including those of hemoglobin-based oxygen carriers combined with freeze-dried plasma.

FIGS. 16A-16G are histograms of complete blood count and hemoglobin concentrations employing various embodiments of the invention and the prior art (WB).

### DETAILED DESCRIPTION OF THE INVENTION

The invention is generally directed to a method of forming a therapeutic hemoglobin-based oxygen carrier solution, a therapeutic hemoglobin-based oxygen carrier solution formed by the method of the invention, and to a method of treating bleeding or anemia and simultaneously increasing systemic convective oxygen delivery of a subject suffering low circulatory oxygen transport or bleeding by administration of the hemoglobin-based oxygen carrier solution of the invention.

In one embodiment the invention is a method of forming a therapeutic hemoglobin-based oxygen carrier solution. The method includes the step of directly combining at least one of freeze-dried platelets and freeze-dried plasma with a hemoglobin-based oxygen carrier to form a therapeutic hemoglobin-based oxygen carrier solution of the invention. “Directly combining,” as that term is used herein, means a hemoglobin-based oxygen carrier (HBOC), such as a hemoglobin-based oxygen carrier, is directly mixed with at least one of freeze-dried platelets and freeze-dried plasma without first rehydrating the freeze-dried platelets or freeze-dried plasma.

In one embodiment, the hemoglobin-based oxygen carrier includes a concentration of calcium chloride of greater than 0.03 mMol/L, a concentration of N-acetyl cysteine of greater than 0.31 mMol/L, a concentration of sodium chloride of less than 76 mMol/L, a concentration of potassium chloride of less than 2.7 mMol/L, a sodium hydroxide concentration of less than 8.3 mMol/L, and a concentration of sodium lactate of less than 18.1 mMol/L. In another embodiment, the hemoglobin-based oxygen carrier includes a concentration of polymerized hemoglobin (>6.0 g/dL and average MW between 130 and 2,000 kD), a concentration of calcium chloride of greater than 0.47 mMol/L, a concentration of N-acetyl cysteine of greater than 4.1 mMol/L, a concentration of sodium chloride of less than 38 mMol/L, a concentration of potassium chloride of less than 1.3 mMol/L, a sodium hydroxide concentration of less than 4.2 mMol/L, and a concentration of sodium lactate of less than 9.0 mMol/L. In still another embodiment, the hemoglobin-based oxygen carrier includes a concentration of polymerized hemoglobin (>12.0 g/dL and average MW between 200 and 500 kD), a concentration of calcium chloride of greater than 0.93 mMol/L, a concentration of N-acetyl cysteine of greater than 8.2 mMol/L, a concentration of sodium chloride of less than 2.8 mMol/L, a concentration of potassium chloride of less than 0.1 mMol/L, a sodium hydroxide concentration of less than 0.3 mMol/L, and concentration of sodium lactate of less than 0.7 mMol/L.

In one specific embodiment, the hemoglobin-based oxygen carrier includes polymerized hemoglobin. In one such embodiment, the hemoglobin is polymerized by reaction with at least one member selected from the group consisting of gluteraldehyde, and other dialdehydes including glycolaldehyde, malondialdehyde, succinaldehyde, glutaraldehyde, adipaldehyde, heptanedial, octanedial, 1,9-Nonanedione (2-9 carbon dialdehyde). Preferably, the hemoglobin is polymerized with glutaraldehyde. In another embodiment, the hemoglobin is polymerized at a pH between 5.5 and 7.0. The hemoglobin can be derived, for example, from at least one source from the group consisting of bovine, porcine, human and Arenicola marina (sea worm). Preferably, the hemoglobin is bovine.

In one embodiment, the polymerized hemoglobin is a purified, filtered, stroma-free hemoglobin-based oxygen carrier solution of heat treated bovine hemoglobin that has an average molecular weight range of from about 130-500 kD. In another embodiment, the polymerized hemoglobin is a purified, filtered, stroma-free hemoglobin-based oxygen carrier solution of heat treated bovine hemoglobin that has an average molecular weight range of from about 130-1,000 kD. In another embodiment, the polymerized hemoglobin is a purified, filtered, stroma-free hemoglobin-based oxygen carrier solution of heat treated bovine hemoglobin that has an average molecular weight range of from about 130-3,000 kD. In another embodiment, the polymerized hemoglobin is a purified, filtered, stroma-free hemoglobin-based oxygen carrier solution of heat treated bovine hemoglobin that has an average molecular weight range of from about 130-6,000 kD. In one embodiment, the hemoglobin-based oxygen carrier employed to form the therapeutic hemoglobin-based oxygen carrier solution of the invention contains, in addition to glutaraldehyde-stabilized and polymerized bovine hemoglobin, sodium chloride, sodium hydroxide, potassium chloride, calcium chloride, sodium lactate and N-acetylcysteine. In another embodiment, the hemoglobin-based oxygen carrier contains, in addition to glutaraldehyde-stabilized polymerized bovine hemoglobin, calcium chloride and N-acetylcysteine, wherein sodium chloride, sodium hydroxide, potassium chloride, and sodium lactate are present at subphysiological concentrations or are absent. In still another embodiment, the hemoglobin-based oxygen carrier contains, in addition to glutaraldehyde-stabilized and polymerized bovine hemoglobin, calcium chloride, in which sodium chloride, sodium hydroxide, potassium chloride, sodium lactate exist at subphysiological concentrations or are absent and N-acetylcysteine is absent.

Examples of suitable hemoglobin-based oxygen carriers include, for example, HBOC-201 which is described, for example, in U.S. Pat. Nos. 5,084,558, 5,296,465, 5,618,919, 5,753,616, 5,895,810, 5,905,141, 5,955,581, 6,506,725, 7,553,613, US2009/0137762 A1, U.S. Pat. Nos. 5,955,581A, 5,952,470A, 5,895,810A, 5,691,452A, 5,753,616A, DE69638066D1, NZ534802A. Additional examples include hemoglobin-based oxygen carrier, VIR—HBOC (OxyBridge™) as described in Vandergriff et al.²⁴, hemoglobin-based oxygen carrier, Sanguinate, as described in AU2015238812B2, CA2764872A1 and EP2440239A1 and hemoblobin-based oxygen carrier, OxyVita as described in WO2017201447A1 and WO2008034138A1 and hemoglobin-based oxygen carrier MP4 as described in U.S. Pat. Nos. 8,377,868, 8,609,815B2 and PCT/US2013/032694, the relevant teachings of all of which are incorporated herein by reference in their entirety.

In one specific embodiment the hemoglobin-based oxygen carrier is combined with freeze-dried platelets. In another specific embodiment, the hemoglobin-based oxygen carrier is directly combined with freeze-dried plasma. In still another embodiment, the hemoglobin-based oxygen carrier is directly combined with both freeze-dried platelets and freeze-dried plasma. In one particular embodiment, the freeze-dried plasma and the freeze-dried platelets are derived from human blood. In one embodiment, whether freeze-dried plasma or freeze-dried platelets, the platelets and plasma are stable without refrigeration (e.g., greater than four-month shelf life, at 2-25 degrees Celsius), rapidly (e.g., less than twelve minutes) rehydrated and pathogen-reduced by ≥2.5 log₁₀ fold.

In one embodiment, the volume ratio of HBOC to freeze-dried platelets is in the range of 0.5:1 to 5:1 where one volume of HBOC is 250 ml and one volume of freeze-dried plasma is derived from 250 ml of human platelet-rich plasma.

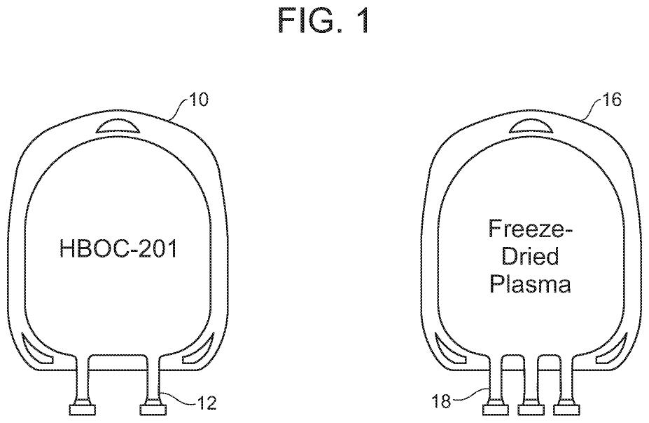

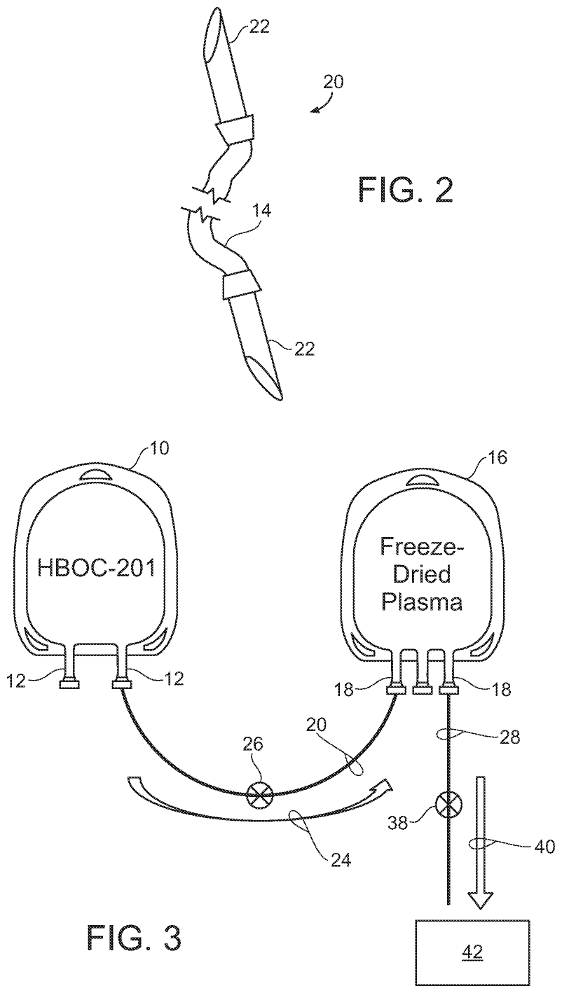

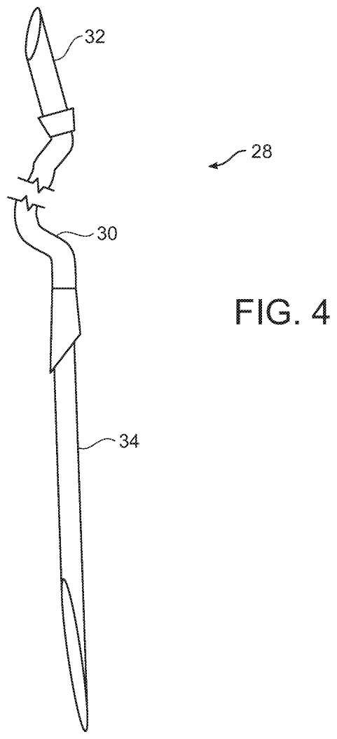

In one embodiment, the hemoglobin solution and at least one of freeze-dried platelets and one of freeze-dried plasma are combined by transferring all of the HBOC solution into one of freeze-dried platelets and inverting the freeze-dried platelets multiple times until platelets are fully rehydrated. The freeze-dried platelets are then transferred to one of freeze-dried plasma and the freeze-dried plasma is inverted multiple times until the freeze dried plasma is fully rehydrated. Alternatively, HBOC may be transferred first to one of freeze-dried plasma before transferring the rehydrated plasma to one of freeze-dried platelets. In one embodiment of a method of the invention of forming a therapeutic hemoglobin-based solution, a system, such as shown in FIGS. 1-6, can be employed. As shown in FIG. 1, hemoglobin-based oxygen carrier (HBOC) container 10 is equipped with ports that each include a membrane located at the base of each of two short tubes of the containers, thereby constituting spike port) 12. Container 16 of freeze-dried plasma (FDP) is equipped with three spike ports 18. FIG. 2 As can be seen in FIG. 2, tube 20 includes a hollow, spiked beveled tip or spike 22 at each end 24, 26, of tubing link 14. Spikes 22 are forced through the membrane located in the spike ports 12, 18 of containers 10, 16, respectively, creating a leak-proof connection through tube 20 between the containers 10, 16 that allows HBOC solution to flow (2) from the HBOC container 10 into the FDP container 16 so connected, as indicated by arrow 24 in FIG. 3. Each transfer tube is also equipped with a releasable clamp or valve 26 allowing closure or opening of 20. Infusion line 28, shown in greater detail in FIG. 4, includes flexible tubing 30 equipped with a spike 32 of design similar to spikes 22 at one end and, on the other end, a large-bore hypodermic needle 34 to gain access to the lumen of a large peripheral blood vessel or intrasseous space of a subject 36 being treated by the method of the invention. Releasable clamp 38 or valve can be employed at tubing 30 to control rate of flow of solution to subject 36 in the direction of arrow 40.

The spikes and the ends of the transfer tubes are inserted into the spike ports 12, 18 on HBOC container 10 and FDP containers 16, respectively, causing transfer of the HBOC solution from HBOC container 10 into FDP container 16. Transfer tube 20 is then clamped closed and the combined FDP and HBOC are mixed by inverting FDP container 16 multiple times until the FDP is completely solubilized and thereby rehydrated. At this point the HBOC-FDP solution is administered to subject 42 through infusion line 28, FIG. 3.

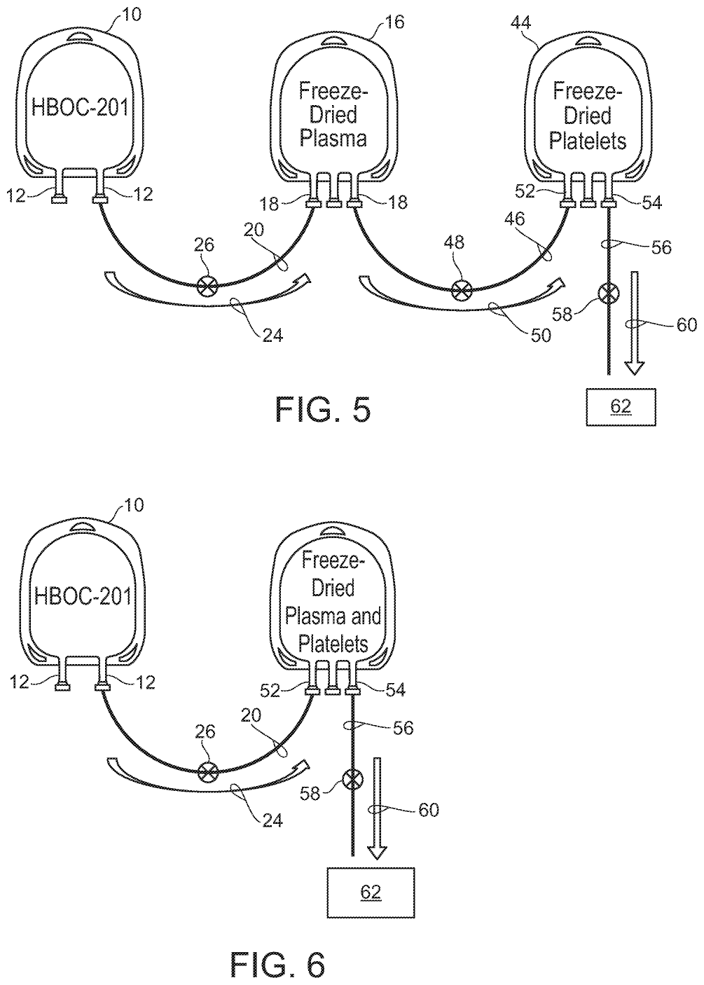

In one embodiment, shown in FIG. 5, all rehydrated contents in FDP container 16 are transferred in direction 50 into non-hydrated FDPlt container 44 through line 46 and clamp or valve 48 linking spike ports 18, 52 on each of containers 16, 44, respectively. Transfer tubing 46 used to effect this transfer is then clamped or shut off by clamp or valve 48 and FDPlt container 44 is inverted multiple times until the contents are fully rehydrated. The resulting HBOC-FDP-FDPlt solution could then be administered through spike port 54, line 56 and clamp or valve 58 in direction 60 to subject 62.

In another embodiment, shown in FIG. 6, HBOC is transferred to container 44 of FDP and FDPlt in a manner as described above for transferring HBOC to a container 16 of FDP alone. The fully rehydrated and mixed HBOC-FDP-FDPlt solution is then be administered to subject 62 via the infusion line 56 as described above.

In another embodiment, the invention is a therapeutic hemoglobin-based oxygen carrier solution formed by a method comprising the step of combining at least one of freeze-dried platelets and freeze-dried plasma with the hemoglobin-based oxygen carrier to form the therapeutic hemoglobin-based solution of the invention, the various embodiments of which are described above.

A method of treating bleeding or anemia and simultaneously increasing systemic and convective oxygen delivery in a subject suffering a low circulatory oxygen transport or bleeding, in one embodiment, includes the step of administering to the subject a therapeutically-effective amount of a therapeutic hemoglobin-based oxygen carrier solution of the invention described above. In one embodiment, the method of treatment includes administration of the therapeutic hemoglobin-based solution intravenously. In another embodiment, the therapeutic hemoglobin-based solution is administered intra-arterially. In still another embodiment, the therapeutic hemoglobin-based solution is administered intraosseously.

In still another embodiment, the subject to which the therapeutic hemoglobin-based solution is administered is in need of treatment by the method of the invention as a consequence of at least one member of the group consisting of ischemia, hypoxia and acute bleeding.

In one embodiment the hypoxia or ischemia is due to at least one member of the group consisting of circulatory hypovolemia, anemia, poor cardiac function, poor pulmonary function, vascular occlusion and vasoconstriction. In one embodiment, the vascular occlusion is due to vascular disease. In one specific embodiment the vascular disease is vascular thrombosis.
In another embodiment, the bleeding is due to at least one member of the group consisting of blunt or penetrating trauma, depletion of platelets, depletion or coagulation factors, dilution of platelets, dilution of coagulation factors, bone marrow disease, liver injury, and liver disease. In yet another embodiment, the depletion of at least coagulation factors is due to consumption of these blood components in the subject due to injury or tissue damage.

While example embodiments have been particularly shown and described, it will be understood by those skilled in the art that various changes in form and details may be made therein without departing from the scope of the embodiments encompassed by the appended claims.

### EXEMPLIFICATION

### Example I

Experiments in Example I were conducted using Standard HBOC-201 (Hemoglobin glutamer-250 [bovine]; Hemopure®, Hemoglobin Oxygen Therapeutics, LLC, Souderton, Pa.) (Std HBOC-201) contained in units sealed and overwrapped at the time of manufacture to exclude oxygen and prevent evaporation. The HBOC-201 was tested for compliance with product release specifications prior to conducting experiments. Testing indicated that HBOC-201 starting product met manufacturing release specifications for all parameters including molecular weight range, average molecular weight, methemoglobin (<5%), oxygenated fraction, hemoglobin concentration and P₅₀. Resuscitation solutions for in vitro testing were formulated by reconstituting freeze-dried plasma (BioPlasma FDP, National Biologics Institute, Pinetown, South Africa) with (A) water (50 ml, the same volume of human plasma from which FDP was derived) or (B) Std HBOC-201 (50 ml). Hemorrhagic resuscitation was simulated in vitro by replacing either 10% (0.5 mL) or 50% (2.5 mL) of a 5-ml volume of fresh whole blood (WB) with the resuscitation solutions, A or B. Undiluted WB served as control.

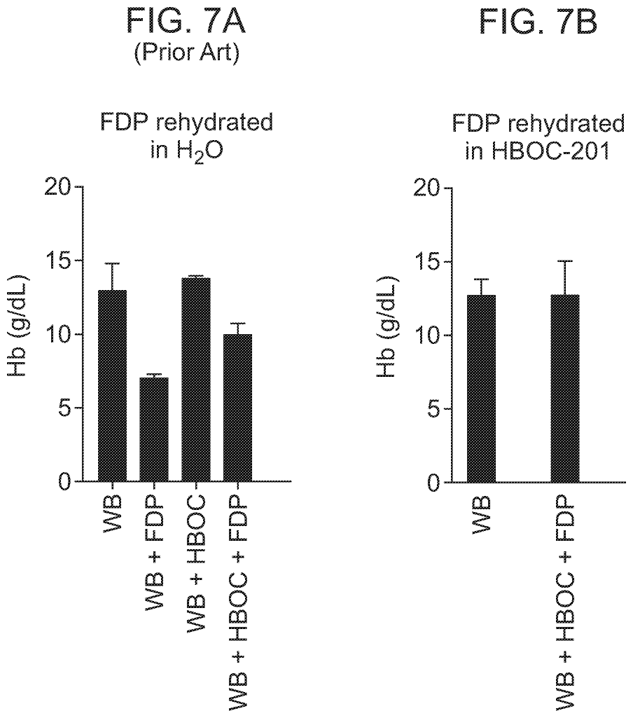

The results of Example I experiments are reported in FIGS. 7-13. These in vitro experiments demonstrate the expected 50% decrease in Hb concentration associated with diluting whole blood (WB) 50% with water-rehydrated FDP (FIG. 7, left panel, WB vs. WB+FDP). Adding the same volume of HBOC-201 (13 g Hb/dL) to WB maintains a normal total Hb concentration (WB vs. WB+HBOC). Replacing 50% of the WB with equal parts of water-rehydrated FDP (25% of total final volume) and HBOC (25% of total final volume) yields an intermediate total Hb concentration (approximately 75% of the Hb concentration in undiluted WB) (FIG. 7, left panel, WB vs. WB+HBOC+FDP). Diluting WB with HBOC-rehydrated FDP allows introduction of plasma coagulation factors while maintaining a normal total Hb concentration (FIG. 7, right panel, WB vs. WB+HBOC+FDP), demonstrating a benefit of this invention.

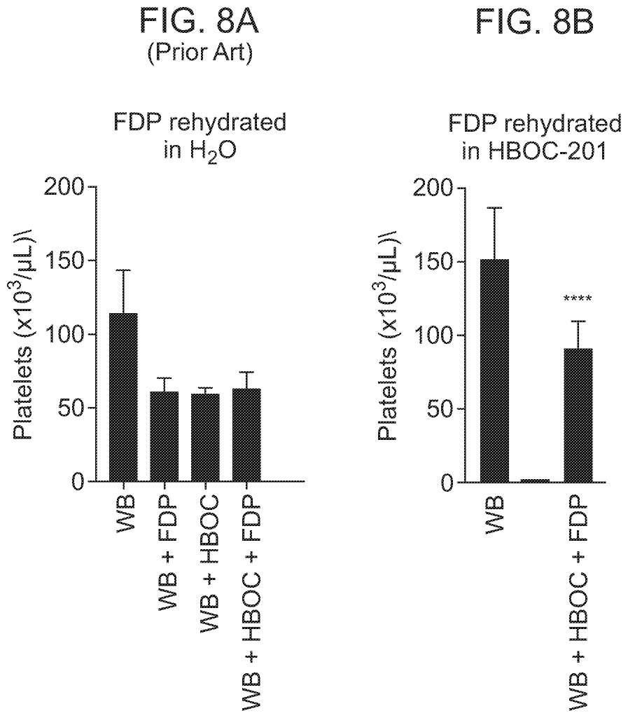

A 50% dilution of WB by water-rehydrated FDP (WB+FDP) or HBOC (WB+HBOC) or HBOC plus water-rehydrated FDP (WB+HBOC+FDP) results in the expected 50% reduction in platelet concentration under all conditions since neither HBOC-201 nor FDP contain platelets (FIG. 8, left panel). Diluting WB 50% with HBOC-rehydrated FDP also yields ˜50% platelet dilution, as expected (FIG. 8, right panel). Rehydration of FDP with HBOC-201 was accomplished by directly combining HBOC-201 with freeze-dried plasma

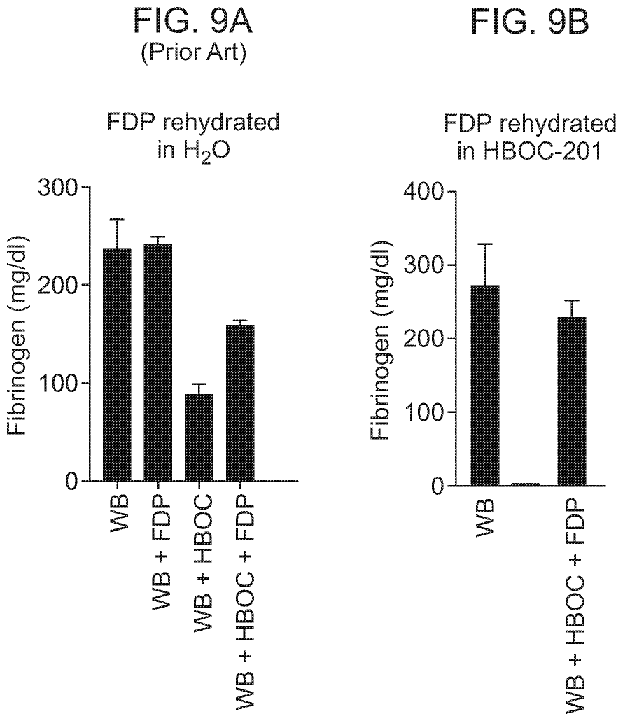

The effects on fibrinogen concentration of WB dilution by water-rehydrated FDP and HBOC-rehydrated FDP are shown in FIG. 9. A 50% replacement of WB by equal parts of a mixture of water-rehydrated FDP (25% of total final volume) and HBOC (WB+HBOC+FDP) (25% of total final volume) reduced fibrinogen concentration by 35% (left panel). The same 50% volume replacement of WB by HBOC-rehydrated FDP (WB+HBOC+FDP) reduced fibrinogen by only 10% (right panel).

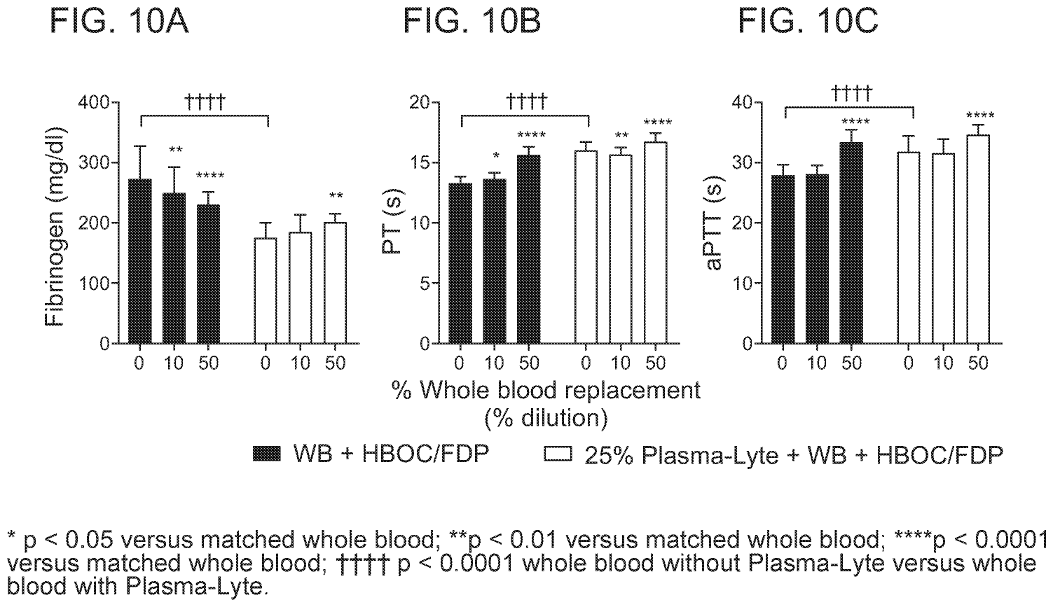

FIG. 10 shows the effects on fibrinogen concentration, prothrombin time (PT) and activated partial thromboplastin time (aPTT) of diluting WB dose-dependently (0, 10 and 50% volume replacement) with HBOC-rehydrated FDP (black bars). PT was extended slightly in a dose-depend fashion, consistent with the slight dose-dependent reduction in fibrinogen concentration. aPTT was also slightly extended by 50% WB dilution, but was unaffected by 10% dilution.

FIG. 10 also shows the effects on fibrinogen concentration, PT and aPTT of further diluting WB that had been previously diluted 25% with Plasma-Lyte, a resuscitation agent lacking Hb, coagulation factors and platelets (gray bars). Prior dilution of WB with Plasma-Lyte simulates a scenario in which an asanguineous crystalloid or colloid resuscitation fluid replaces blood loss by a hemorrhagic trauma patient prior to administration of an HBOC-FDP combination product. Dilution of WB with Plasma-Lyte further diluted fibrinogen concentration and increased PT and aPTT as expected. Further dilution (10% and 50%) of the WB+Plasma-Lyte mixture with HBOC-rehydrated FDP dose-dependently increased fibrinogen concentration and hence, produced smaller increases in PT and aPTT than occurred with these dilutions in undiluted WB. These results demonstrate the ability of the HBOC-rehydrated FDP to minimize dilutional coagulopathy.

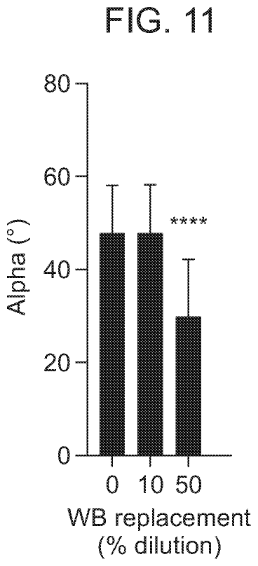

FIG. 11 shows the effect of WB replacement by HBOC-rehydrated FDP on TEG alpha angle (a measure of the rate at which clot size increases). A 50% replacement of WB resulted in a 40% reduction in alpha angle, a result that was at least qualitatively expected based on the known impacts of platelet dilution and cell-free Hb on coagulation, fibrinolysis and platelet function reported in the literature and described earlier. However, 10% WB dilution had no effect on alpha angle.

The collective results of replacing WB and WB-Plasma-Lyte with HBOC-rehydrated FDP on PT and aPTT (FIG. 10) and on TEG alpha angle (FIG. 11) suggest the possibility that the component(s) in HBOC responsible for extended PT and aPTT times and decreased alpha angle may have a therapeutically relevant threshold concentration, below which platelet and coagulation inhibitory effects described in the literature fail to manifest. This observation suggests that the invention may, unexpectedly, have an ability to intrinsically overcome the obstacle (inhibition of platelet function and coagulation by components in HBOC) described by the scientific literature. Specifically, HBOC solution components may exist in the combination product package at concentrations that inhibit clot propagation in unadulterated samples of the combination product, but function normally or nearly so when diluted upon intravenous infusion in the treated patient, thereby unmasking the clinical benefit (simultaneous support of circulatory volume, total hemoglobin concentration and hemostasis) of this innovation. Assuming a typical initial HBOC-FDP treatment dose of 500 ml, the solution in the delivery package after FDP and/or FDPlt rehydration will be diluted by approximately 90% when infused into a patient having a 5 L blood volume.

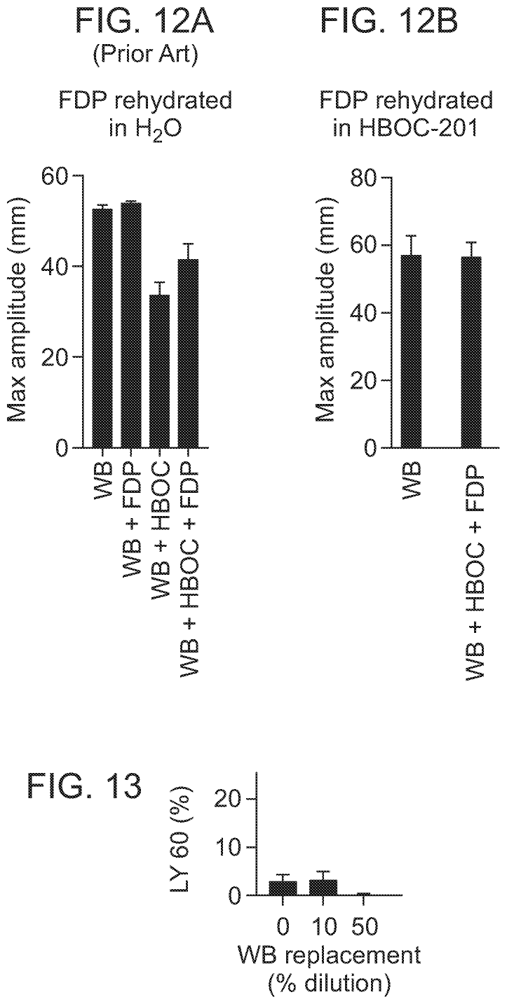

FIG. 12 shows that TEG parameter, MA, is depressed by 50% replacement of WB with equal parts of water-rehydrated FDP (25% of total final volume) plus HBOC (25% of total final volume) added separately (left panel, WB vs. WB+HBOC+FDP), but is unaffected when WB is replaced 50% by HBOC-rehydrated FDP (right panel). These results indicate that replacing WB with HBOC-rehydrated FDP is capable of overcoming the dilution of coagulation factors and platelets associated with separately adding HBOC and water-rehydrated FDP to WB. Thus, although the rate of clot formation (FIG. 11, Alpha angle) is inhibited by 50% replacement of WB by HBOC-rehydrated FDP, normal clot size was, surprisingly, still achieved. From a clinical perspective, clot size and strength are the ultimate determinants of hemostasis in hemorrhagic trauma patients. ^{25, 26}

FIG. 13 shows the effects of replacing WB with an HBOC-rehydrated FDP on extent of clot lysis 60 min following inititation of the clotting reaction. Ten percent WB replacement had no effect on the rate of clot lysis compared to control (0% replacement of WB) and lysis tended to be slower than control when 50% of WB was replaced by HBOC-rehydrated FDP. This result is contrary to reports describing HBOC-induced stimulation of thrombolysis via a tPA-independent mechanism as discussed above.⁴ Although replacing WB with HBOC-rehydrated FDP eliminated dilution-induced acceleration of thrombolysis, the greatest replacement (50%) of WB by HBOC-rehydrated FDP should have enhanced tPA-independent thrombolysis over that observed in undiluted WB and in WB replaced 10%. These data suggest that the clot formed under these test conditions has significant strength, enabling the clot to resist thrombolysis and/or that thrombolytic activity is unaffected (10% WB replacement) or even directly inhibited (50% WB replacement) by the tested HBOC-rehydrated FDP solution.

Our results show that normal clot size can be achieved by clinically massive blood replacement (up to and including 50%) of WB by a solution of HBOC-rehydrated FDP and that thrombolysis is either unaffected or even inhibited by HBOC+FDP, providing additional unexpected evidence that the claimed invention (treating anemia, coagulopathy and hypovolemia via infusion of an HBOC-rehydrated FDP solution) can, in a simulated in vivo application, overcome the negative impact of HBOC on fibrinolysis, coagulation and platelet function described in the scientific literature.

FIGS. 7A and 7B show the effect of 50% volume replacement of whole blood on Hb concentration: H₂O-rehydrated FDP vs. HBOC-rehydrated FDP.

As can be seen therein, FIG. 7A shows H₂O-rehydrated FDP (WB+FDP), HBOC-201 (WB+HBOC) or HBOC-201 (25% of total final volume) plus H₂O-rehydrated FDP (WB+HBOC+FDP) (25% of total final volume) were added to human whole blood (WB) to achieve 50% WB replacement. (Control=WB with no addition (WB).)

FIG. 7B shows that, when HBOC-201-rehydrated FDP (WB+HBOC+FDP) was added to human whole blood (WB), 50% volume replacement was achieved. (Control=WB with no addition (WB).)

(FDP=human freeze-dried plasma; WB=human whole blood.)

FIGS. 8A and 8B show the effect of 50% whole blood volume replacement on platelet concentration: H₂O-rehydrated FDP vs. HBOC-rehydrated FDP.

As can be seen in FIG. 8A, H₂O-rehydrated FDP (WB+FDP), HBOC-201 (WB+HBOC) or HBOC-201 (25% of total final volume) plus H₂O-rehydrated FDP (WB+HBOC+FDP) (25% of total final volume) were added to WB to achieve a 50% WB volume replacement. Control=WB with no addition (WB).

As can be seen in FIG. 8B, HBOC-201-rehydrated FDP (WB+HBOC+FDP) were added to WB to achieve 50% WB volume replacement. (Control=WB with no addition (WB).)

(FDP=human freeze-dried plasma; WB=human whole blood.)

FIGS. 9A and 9B show the effect of 50% whole blood volume replacement on fibrinogen concentration: H₂O-rehydrated FDP vs. HBOC-rehydrated FDP.

As can be seen in FIG. 9A, H₂O-rehydrated FDP (WB+FDP), HBOC-201 (WB+HBOC) or HBOC-201 (25% of total final volume) plus H₂O-rehydrated FDP (WB+HBOC+FDP) (25% of total final volume) were added to WB to achieve 50% WB replacement. (Control=WB with no addition (WB).)

As can be seen in FIG. 9B, HBOC-201-rehydrated FDP (WB+HBOC+FDP) was added to WB to achieve 50% WB replacement. (Control=WB with no addition (WB).)

(FDP=human freeze-dried plasma; WB=human whole blood. FDP=human freeze-dried plasma; WB=human whole blood.)

FIG. 10 shows the effect of dose-dependent dilution of whole blood by HBOC-rehydrated FDP on fibrinogen concentration, prothrombin time and activated partial thromboplastin time.

Black bars in FIGS. 10A-10C indicate where HBOC-201-rehydrated FDP (WB+HBOC/FDP) was added to WB to achieve a 10% or 50% WB volume replacement. (Control=WB with no addition (0% replacement).)

Gray bars in FIGS. 10A-10C indicate where HBOC-201-rehydrated FDP (WB+HBOC/FDP) was added to WB that had been previously diluted 25% with Plasma-Lyte (resuscitation fluid lacking Hb, coagulation factors and platelets) (Baxter International, Inc., Deerfield, Ill.) to simulate replacement of lost blood by an asanguineous crystalloid or colloid resuscitation fluid prior to administration of an HBOC-FDP product. (Control=WB with no addition (0% replacement with HBOC-rehydrated FDP).)

(PT=prothrombin time; aPTT=activated partial thromboplastin time; FDP=human freeze-dried plasma; WB=human whole blood.)

(*P<0.01 vs. control; **P<0.01 vs. control; ****P<0.0001 vs. control (middle panel); ****P<0.0001 control WB vs. control Plasma-Lyte+WB (right panel).)

FIG. 11 shows the effect of whole blood replacement by HBOC-rehydrated FDP on TEG alpha angle.

As can be seen in FIG. 11, HBOC-201-rehydrated FDP (WB+HBOC/FDP) added to WB to achieve a 10% or 50% volume replacement. (Control=WB with no addition (0% replacement). Alpha=angle)(° of tangent to clot edge at a clot diameter of 20 mm and is a measure of clot formation rate.)

(FDP=human freeze-dried plasma; WB=human whole blood.

****P<0.0001 vs. control.)

FIGS. 12A-12B show the effect of of 50% volume replacement of whole blood on TEG maximum amplitude: FDP in H₂O vs. FDP in HBOC.

FIG. 12A shows the effect of H₂O-rehydrated FDP (WB+FDP), HBOC-201 (WB+HBOC) or HBOC-201 (25% of total final volume) plus H₂O-rehydrated FDP (25% of total final volume) (WB+HBOC+FDP) were added to WB to achieve 50% WB volume replacement. (Control=WB with no addition (WB).)

FIG. 12B shows the effect of HBOC-201-rehydrated FDP (WB+HBOC+FDP) was added to WB to achieve a 50% WB volume replacement. (Control=WB with no addition (WB).)

(FDP=human freeze-dried plasma; WB=human whole blood.)

FIG. 13 shows the effect of 50% WB volume replacement by HBOC-rehydrated FDP on TEG clot lysis 60 min after initiation of the clotting reaction.

HBOC-201-rehydrated FDP was added to WB to achieve 10% or 50% volume replacements. Control=WB with no addition (0% replacement). LY60(%) is the percent of the clot that has lysed 60 min after initiation of the clot reaction.

(FDP=human freeze-dried plasma; WB=human whole blood.)

### Example II

Studies were conducted to extend the initial work (described in Example I) in which water-rehydrated freeze-dried plasma (FDP) (BioPlasma FDP, National Biologics Institute, Pinetown, South Africa) and standard HBOC-201-rehydrated FDP were compared with respect to thromboelastography (TEG), aPTT, PT, fibrinogen concentration and complete blood cell count. The work described in Example I was extended by rehydrating FDP with one of a series of HBOCs having varied concentrations low MW components (<30 kD) as described below in Methods.

### Methods

Standard HBOC-201 (Hemoglobin glutamer-250 [bovine]; Hemopure®, Hemoglobin Oxygen Therapeutics, LLC, Souderton, Pa.) (Std HBOC-201) was diafltered (DiFi-HBOC) to reduce components having a MW below 30 kD. Low MW components reduced in DiFi-HBOC included lactate, sodium, potassium, chloride, calcium (C), hydroxide ion and N-acetyl cysteine (NAC, N). HBOC-201 diafiltrations were also conducted to separately and dually retain calcium and NAC at the original concentrations in the retentate solution. Prior to diafiltration, HBOC-201 was tested for compliance with manufacturing release specifications. Testing indicated that HBOC-201 starting product met release specifications for all parameters including molecular weight range, average molecular weight, methemoglobin (<5%), oxygenated fraction, hemoglobin concentration and Pso. HBOC-201 was diafiltered at room temperature under oxygen-free conditions across a 30 kD cut-off membrane against five exchanges of the replacement buffer to achieve the various targeted diafiltered HBOC solution profiles. Low-molecular weight components (calcium chloride and/or N-acetyl cysteine (NAC)) to be retained in the retentate were included in the replacement buffer at their original concentrations in HBOC-201. The resulting HBOCs are identified and defined in Table 1.

TABLE 1
Low MW components of HBOCs created
via diafiltration of HBOC-201

| | | HBOC designation | | | |
|---|---|---|---|---|---|
| HBOC | HBOC- | DiFi- | DiFi- | DiFi- | DiFi- |
| component | 201 | HBOC | HBOC-C | HBOC-N | HBOC-N-C |
| abbreviated | Component concentration | | | | |
| name | (mMol/L) | | | | |
| NaCl | 114 | 2.8 | 2.8 | 2.8 | 2.8 |
| KCl | 4 | 0.1 | 0.1 | 0.1 | 0.1 |
| CaCl2 | 1.4 | 0.0 | 1.4 | 0.0 | 1.4 |
| NaOH | 12.5 | 0.3 | 0.3 | 0.3 | 0.3 |
| Na lactate | 27.1 | 0.7 | 0.7 | 0.7 | 0.7 |
| NAC | 12.3 | 0.3 | 0.3 | 12.3 | 12.3 |

NaCl = sodium chloride, KCl = potassium chloride, NaOH = sodium hydroxide, Na lactate = sodium lactate, NAC = N-acetyl-L-cysteine. In HBOC-201 and all of the HBOCs listed above, hemoglobin concentration = 13 ± 1 g/dL, pH = 7.6-7.9 and metHb is <5%.

Thus, solutions used to rehydrate FDP included (A) water, (B) Std HBOC-201, (C) DiFi-HBOC with all <30 kD components reduced, (D) DiFi-HBOC—N with <30 kD components reduced except NAC, (E) DiFi-HBOC—C with <30 kD components reduced except calcium, or (F) DiFi-HBOC—N—C with <30 kD components reduced except NAC and calcium. Conductivity tests before and after completion of the diafiltration process indicated that >97.5% of all low-molecular weight components targeted for elimination had been excluded from the final retentate and that the final total hemoglobin concentration (13±1 g/dL) and pH (7.6-7.9) in all HBOCs were equivalent to those of the starting HBOC-201 solution.

Resuscitation solutions for in vitro testing were formulated by reconstituting freeze-dried plasma (BioPlasma FDP, National Biologics Institute, Pinetown, South Africa) with one of the solutions A-F (50 ml, the volume of human plasma from which the FDP was derived). In all cases involving rehydration of FDP by HBOC-201 or HBOC employed, the HBOC-201 or HBOC was directly combined with FDP. Hemorrhagic resuscitation was simulated in vitro by replacing either 10% (0.5 mL) or 50% (2.5 mL) of a 5-ml volume of fresh whole blood (WB) with the various resuscitation solutions, A-F. Undiluted WB served as control. Since tissue plasminogen activator (tPA) is released from endothelium in ischemic shock, resulting in a fibrinolytic phenotype facilitating vessel patency, tPA was added to duplicates of all experimental assay mixtures to model hemorrhagic shock-induced fibrinolysis.

### Results

### Thromboelastography

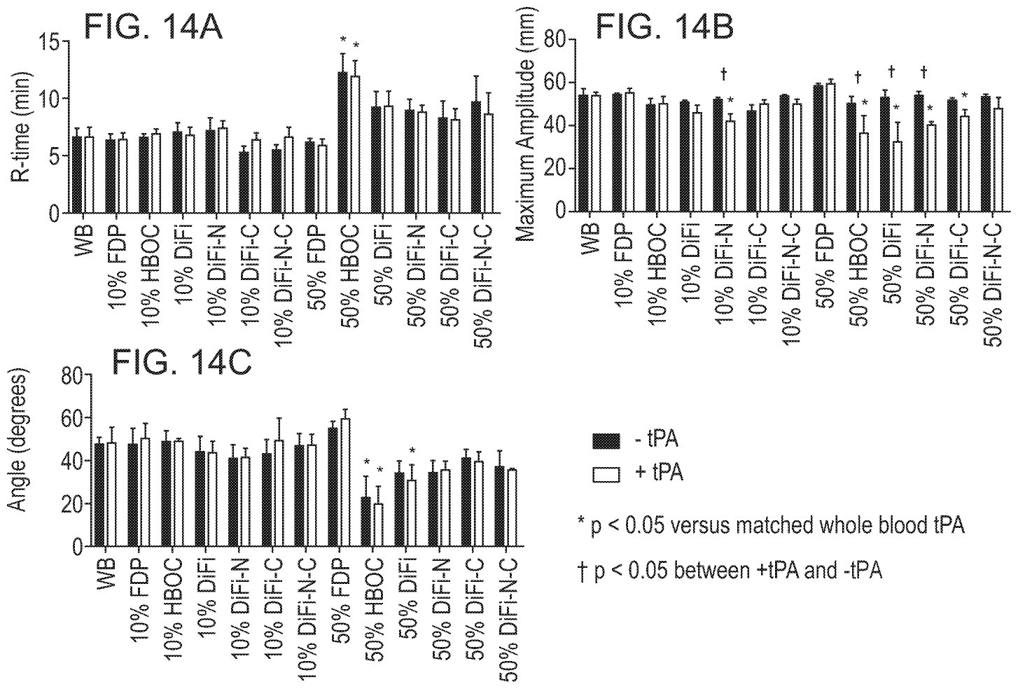

The results of Example II experiments are reported in FIGS. 14-16. 50% dilution of whole blood (WB) with standard (Std) HBOC-201 increased R-time and decreased angle, effects that were mitigated with all HBOC formulations (FIGS. 14A-14C).

Water-rehydrated and Std HBOC-201-rehydrated FDP had little effect on maximum amplitude (MA), a measure of clot strength, and all HBOCs preserved MA, even at 50% WB replacement (FIG. 14B).

Fibrinolysis at 60 min (LY60) was negligible without tPA, which induced significant changes when WB was replaced 10% by FDP rehydrated with DiFi-HBOC-201 or DiFi-HBOC—N, both HBOCs lacking calcium (23.6±7.3% and 26.6±8.6%, respectively, versus 6.7±4.1% in WB; p<0.001 for each comparison) (FIG. 14E). Replacing WB by 10% with FDP rehydrated with HBOCs DiFi-HBOC—C containing calcium or DiFi-HBOC—N—C calcium plus NAC substantially mitigated fibrinolysis (FIG. 14E). Similar calcium-dependent patterns were observed with 50% WB replacement by HBOCs at 60 min (FIG. 14E) and 30 min (FIG. 14D) after initiating the clotting process.

WB: Whole blood only.
tPA: Tissue plasminogen activator.
FDP: Freeze-dried plasma.
10% FDP: 10% vol replacement of WB with FDP rehydrated with water.
10% HBOC: 10% vol replacement of WB with FDP rehydrated with Std HBOC-201.
10% DiFi: 10% vol replacement of WB with FDP rehydrated with DiFi-HBOC.
10% DiFi-N: 10% vol replacement of WB with FDP rehydrated with DiFi-HBOC containing NAC.
10% DiFi-C: 10% vol replacement of WB with FDP rehydrated with DiFi-HBOC containing calcium.
10% DiFi-N—C: 10% vol replacement of WB with FDP rehydrated with DiFi-HBOC containing NAC+calcium.
50% FDP: 50% vol replacement of WB with FDP rehydrated with water.
50% HBOC: 50% vol replacement of WB with FDP rehydrated with Std HBOC-201.
50% DiFi: 50% vol replacement of WB with FDP rehydrated with DiFi-HBOC.
50% DiFi-N; 50% vol replacement of WB with FDP rehydrated with DiFi-HBOC containing NAC.
50% DiFi-C: 50% vol replacement of WB with FDP rehydrated with DiFi-Hemopure containing calcium.
50% DiFi-N—C: 50% vol replacement of WB with FDP rehydrated with DiFi-Hemopure containing NAC+calcium.

* p<0.05 versus matched whole blood; t p<0.05 (significant difference) between +tPA and—tPA.

### Coagulation and Fibrinogen Assays

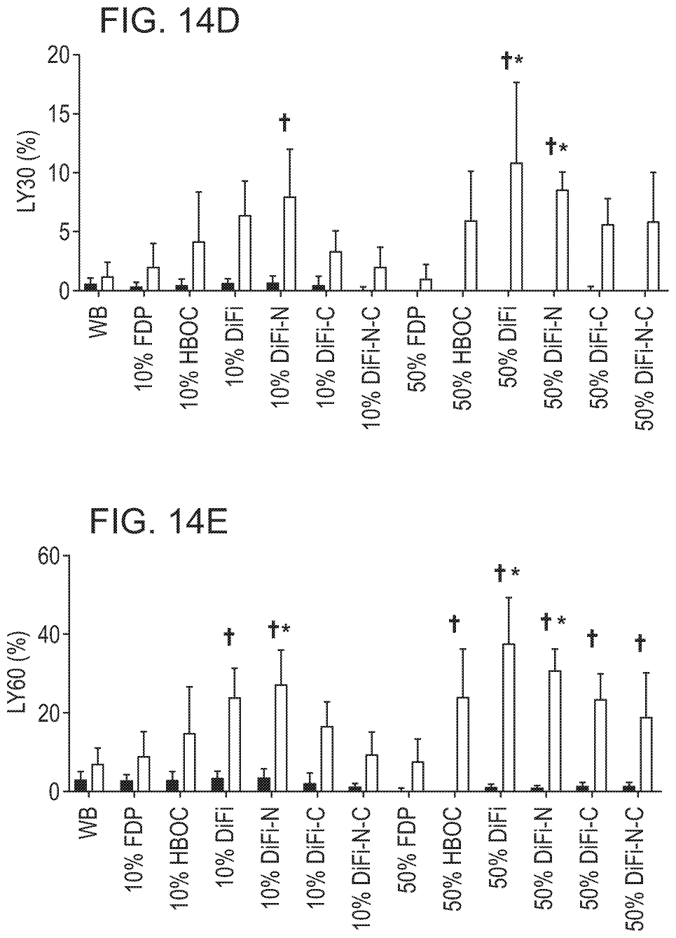

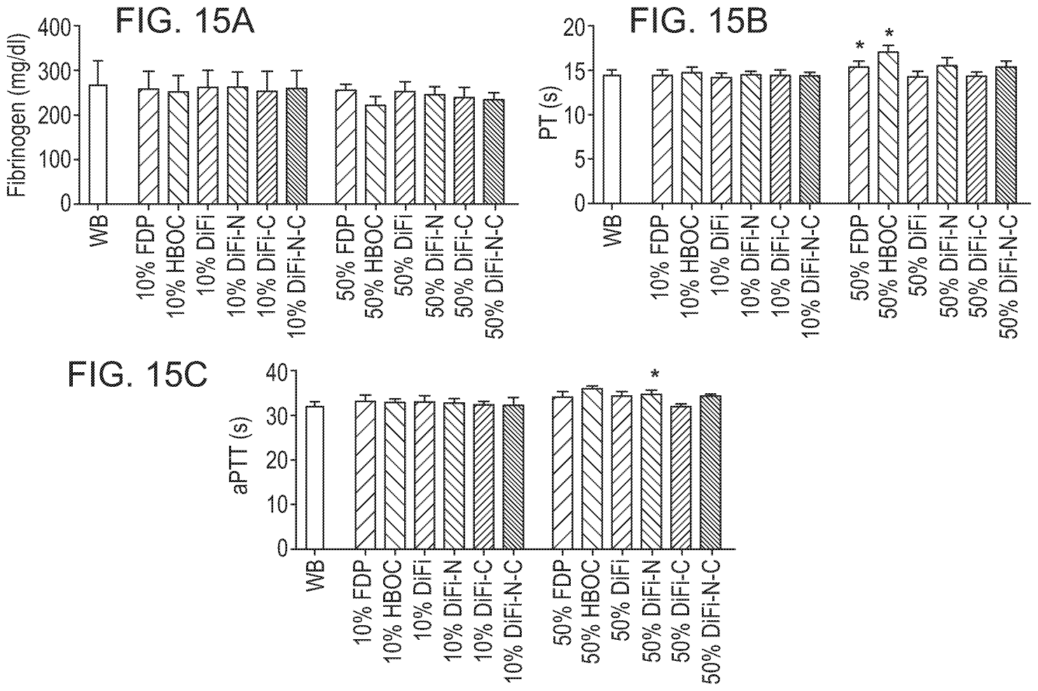

No significant changes were observed in fibrinogen concentration (FIG. 15A). Slight increases in WB PT (14.6±0.5 s) caused by 50% replacement of WB with water-rehydrated FDP (15.5±0.6 s, p<0.05 vs. WB) or HBOC-201-rehydrated FDP (17.27±0.6 s, p<0.05 vs. WB) were mitigated by rehydrating FDP with HBOC DiFi-HBOC-201 retaining calcium (FIG. 15B). A similar treatment-dependent profile was observed in 50% WB replacement samples for aPTT (FIG. 15C).
WB: Whole blood
aPTT: Activated partial thromboplastin time.
PT: Prothrombin time.
FDP: Freeze-dried plasma.
10% FDP: 10% vol replacement of WB with FDP rehydrated with water.
10% HBOC: 10% vol replacement of WB with FDP rehydrated with Std HBOC-201.
10% DiFi: 10% vol replacement of WB with FDP rehydrated with DiFi-HBOC.
10% DiFi-N: 10% vol replacement of WB with FDP rehydrated with DiFi-HBOC containing NAC.
10% DiFi-C: 10% vol replacement of WB with FDP rehydrated with DiFi-HBOC containing calcium.
10% DiFi-N—C: 10% vol replacement of WB with FDP rehydrated with DiFi-HBOC containing NAC+calcium.
50% FDP: 50% vol replacement of WB with FDP rehydrated with water.
50% HBOC: 50% vol replacement of WB with FDP rehydrated with Std HBOC-201.
50% DiFi: 50% vol replacement of WB with FDP rehydrated with DiFi-HBOC.
50% DiFi-N: 50% vol replacement of WB with FDP rehydrated with DiFi-HBOC containing NAC.
50% DiFi-C: 50% vol replacement of WB with FDP rehydrated with DiFi-Hemopure containing calcium.
50% DiFi-N—C: 50% vol replacement of WB with FDP rehydrated with DiFi-Hemopure containing NAC+calcium.
* p<0.05 versus matched whole blood.

### Complete Blood Count and Hemoglobin Concentration

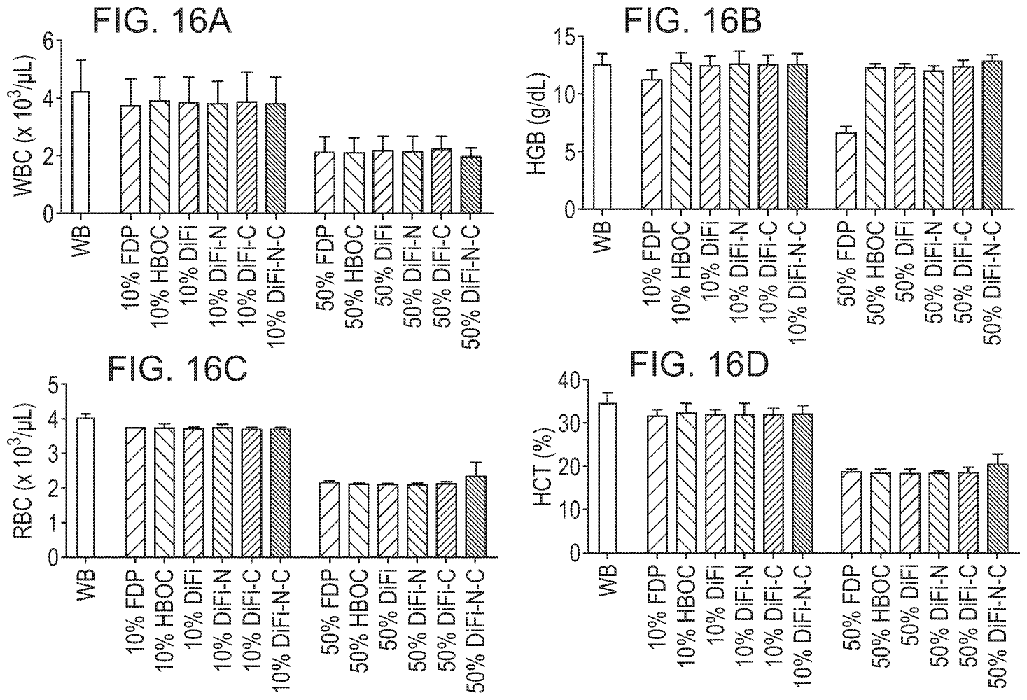

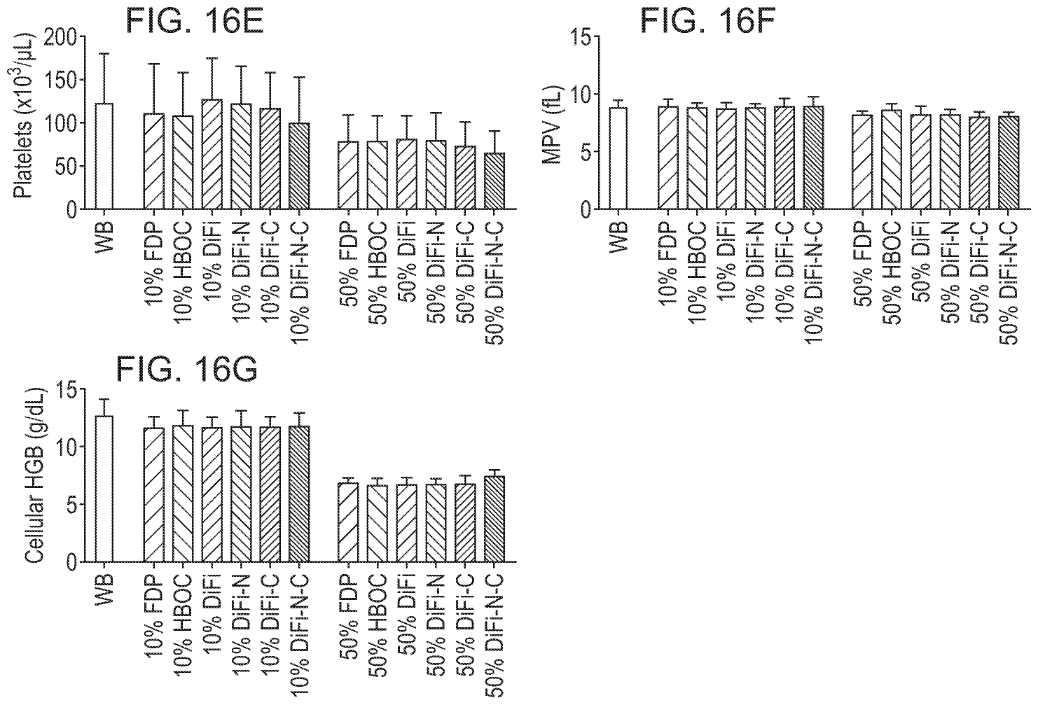

Partial replacement of WB with FDP rehydrated with water or any of the HBOC formulations decreased cell counts proportional to the replacement volume, consistent with an expected dilutional effect since neither FDP nor HBOC contain any cells (FIGS. 16A, C, D, E). Partial replacement of WB with FDP rehydrated with any of the HBOC formulations maintained total hemoglobin concentrations (HGB) in a range similar to that of WB (FIG. 16B). RBC-specific hemoglobin concentration (cellular HGB), however, decreased with partial WB replacement by all HBOC-rehydrated FDP formulations, as expected (FIG. 16G).
tPA: Tissue plasminogen activator
FDP: Freeze-dried plasma.
10% FDP: 10% vol replacement of WB with FDP rehydrated with water.
10% HBOC: 10% vol replacement of WB with FDP rehydrated with Std HBOC-201.
10% DiFi: 10% vol replacement of WB with FDP rehydrated with DiFi-HBOC.
10% DiFi-N: 10% vol replacement of WB with FDP rehydrated with DiFi-HBOC containing NAC.
10% DiFi-C: 10% vol replacement of WB with FDP rehydrated with DiFi-HBOC containing calcium.
10% DiFi-N—C: 10% vol replacement of WB with FDP rehydrated with DiFi-HBOC containing NAC+calcium.
50% FDP: 50% vol replacement of WB with FDP rehydrated with water.
50% HBOC: 50% vol replacement of WB with FDP rehydrated with Std HBOC-201.
50% DiFi: 50% vol replacement of WB with FDP rehydrated with DiFi-HBOC.
50% DiFi-N; 50% vol replacement of WB with FDP rehydrated with DiFi-HBOC containing NAC.
50% DiFi-C: 50% vol replacement of WB with FDP rehydrated with DiFi-HBOC containing calcium.
50% DiFi-N—C: 50% vol replacement of WB with FDP rehydrated with DiFi-HBOC containing NAC+calcium.

### Interpretation of Results

Partial replacement of whole blood by HBOCs in which all low molecular weight components of HBOC-201 had been reduced via diafiltration improved TEG R-time and angle compared to Std HBOC-201. This observation suggests that one or more low molecular weight components of HBOC-201 has a deleterious effect on clot formation. Addition of tPA to mixtures of WB and FDP rehydrated with calcium-free formulations of a HBOC resulted in exaggerated clot lysis compared to mixtures of WB and FDP rehydrated with water or Std HBOC-201 containing a physiologic concentration of calcium. By contrast, partial WB replacement by FDP rehydrated with HBOC DiFi-HBOC—C containing calcium sharply reduced thrombolysis. The inclusion of NAC in some HBOC formulations used to rehydrate FDP had little or no effect across all TEG and coagulation assays.

We conclude from these results that overall clotting activity in mixtures of WB and FDP rehydrated with an HBOC is optimized by eliminating all low molecular weight (<30 kD) components of polymerized hemoglobin solutions except calcium and NAC. Low MW components eliminated to create HBOCs in this study (other than calcium and NAC) were sodium, potassium, chloride, hydroxide ion and lactate. One or more of these components appear to negatively impact the clotting process. Calcium is an important regulator of several clotting factors and its presence in HBOC under the conditions of the described TEG assays is an important contributor to the clotting process.²⁷ NAC provides protection against spontaneous oxidation of cell-free hemoglobin in HBOC solutions to maintain a long HBOC shelf life without materially impacting the clotting process.

### REFERENCES

1 Pusateri A E, Given M B, Schreiber M A, Spinella P C, Pati S, Kozar R A, Khan A, Dacorta J A, Kupferer K R, Prat N, Pidcoke H F, Macdonald V W, Malloy W W, Sailliol A, Cap A P. Dried plasma: state of the science and recent developments. Transfusion. 2016 April; 56 Suppl 2:S128-39. doi: 10.1111/trf.13580. Review. PubMed PMID: 27100749.
2 Arnaud F, Fasipe D, Philbin N, Rice J, Flournoy W S, Ahlers S, McCarron R, Freilich D. Hematology patterns after hemoglobin-based oxygen carrier resuscitation from severe controlled hemorrhage with prolonged delayed definitive care. Transfusion. 2007 November; 47(11):2098-109. PubMed PMID: 17958540.
3 Bickell W H, Stern S. Fluid replacement for hypotensive injury victims: how, when and what risks? Curr Opin Anaesthesiol 1998; 11:177-80.
4 Morton A P, Moore E E, Moore H B, Gonzalez E, Chapman M P, Peltz E, Banerjee A, Silliman C. Hemoglobin-based oxygen carriers promote systemic hyperfibrinolysis that is both dependent and independent of plasmin. J Surg Res. 2017 Jun. 1; 213:166-170. doi: 10.1016/j.jss.2015.04.077. Epub 2015 Apr. 25. PubMed PMID: 28601310; PubMed Central PMCID: PMC5467451.
5 Wiman B, Collen D. On the mechanism of the reaction between human alpha 2-antiplasmin and plasmin. J Biol Chem. 1979; 254:9291.
6 Robbie L A, Booth N A, Croll A M, Bennett B. The roles of alpha 2-antiplasmin and plasminogen activator inhibitor 1 (PAI-1) in the inhibition of clot lysis. Thromb Haemost. 1993; 70:301.
7 Cotton B A, Harvin J A, Kostousouv V, et al. Hyperfibrinolysis at admission is an uncommon but highly lethal event associated with shock and prehospital fluid administration. J Trauma Acute Care Surg. 2012; 73:365. discussion 70.
8 Kostousov V, Wang Y W, Cotton B A, Wade C E, Holcomb J B, Matijevic N. Influence of resuscitation fluids, fresh frozen plasma and antifibrinolytics on fibrinolysis in a thrombelastography-based, in-vitro, whole-blood model. Blood Coagul Fibrinolysis. 2013 July; 24(5):489-97. doi: 10.1097/MBC.0b013e32835e4246. PubMed PMID: 23406662.
9 Rugeri L, Levrat A, David J S, Delecroix E, Floccard B, Gros A, Allaouchiche B, Negrier C. Diagnosis of early coagulation abnormalities in trauma patients by rotation thrombelastography. J Thromb Haemost. 2007 February; 5(2):289-95. Epub 2006 Nov. 16. PubMed PMID: 17109736.
10 Bolliger D, Szlam F, Levy J H, Molinaro R J, Tanaka K A. Haemodilution-induced profibrinolytic state is mitigated by fresh-frozen plasma: implications for early haemostatic intervention in massive haemorrhage. Br J Anaesth. 2010; 104:318.
11 Chapman M P, Moore E E, Ramos C R, et al. Fibrinolysis greater than 3% is the critical value for initiation of antifibrinolytic therapy. J Trauma acute Care Surg. 2013; 75:961.
12 Moallempour M, Jahr J S, Lim J C, Weeks D, Butch A, Driessen B. Methemoglobin effects on coagulation: a dose-response study with HBOC-200 (Oxyglobin) in a thrombelastogram model. J Cardiothorac Vasc Anesth. 2009 February; 23(1):41-7. doi: 10.1053/j.jvca.2008.06.006. Epub 2008 Aug. 29. PubMed PMID: 18834828.
13 Jahr J S, Weeks D, Desai P, et al: Does Oxyvita, a new generation hemoglobin-based oxygen carrier, or Oxyglobin acutely interfere with coagulation compared with normal saline or 6% Hetastarch? An ex vivo thromboelastography preliminary study. J Cardiothorac Vasc Anesth 22:34-39, 2008
14 Scott, E. M., and J. C. McGraw. 1962. Properties of diphosphopyridine nucleotide diaphorase of human erythrocytes. J. Biol. Chem. 237: 249-252.
15 Mumby S, Ramakrishnan L, Evans T W, Griffiths M J, Quinlan G J. Methemoglobin-induced signaling and chemokine responses in human alveolar epithelial cells. Am J Physiol Lung Cell Mol Physiol. 2014 Jan. 1; 306(1):L88-100. doi: 10.1152/ajplung.00066.2013. Epub 2013 Oct. 18. PubMed PMID: 24142518; PubMed Central PMCID: PMC3920215.
16 Alayash A I. Blood substitutes: why haven't we been more successful? Trends Biotechnol. 2014 April; 32(4):177-85. doi: 10.1016/j.tibtech.2014.02.006. Epub 2014 Mar. 12. Review. PubMed PMID: 24630491; PubMed Central PMCID: PMC4418436.
17 Foster M W, McMahon T J, Stamler J S. S-nitrosylation in health and disease. Trends Mol Med. 2003; 9:160-168
18 Jensen D E, Belka G K, Du Bois GC. S-Nitrosoglutathione is a substrate for rat alcohol dehydrogenase class III isoenzyme. Biochem J. 1998; 331 (Pt 2):659-668
19 Furie B, Furie B C. Mechanisms of thrombus formation. N Engl J Med. 2008 Aug. 28; 359(9):938-49. doi: 10.1056/NEJMra0801082. Review. PubMed PMID: 18753650.
20 Essex D W, Li M. Redox modification of platelet glycoproteins. Curr Drug Targets. 2006 October; 7(10):1233-41. Review. PubMed PMID: 17073584.
21 Manickam N, Ahmad S S, Essex D W. Vicinal thiols are required for activation of the αIIbβ3 platelet integrin. J Thromb Haemost. 2011 June; 9(6):1207-15. doi: 10.1111/j.1538-7836.2011.04266.x. PubMed PMID: 21645227.
22 Freilich, D A. Multfunctional blood substitute, US Patent 2007/0265195 A1. Published 15 Nov. 2007.
23 Nadler R, Gendler S, Benov A, Shina A, Baruch E, Twig G, Glassberg E. Intravenous access in the prehospital settings: What can be learned from point-of-injury experience. J Trauma Acute Care Surg. 2015 August; 79(2):221-6. doi: 10.1097/TA.0000000000000723. PubMed PMID: 26218689.
24 Vandergriff K D, Song B K, Nugent W H, Light W R. Vir-Tech Bio's progress and future plans with a large human hemoglobin polymer. The 17th Congress of the International Symposium on Blood Substitutes and Oxygen Therapeutics, Nara Kasugano, Japan, 21-23 Nov., 2019
25 Tutwiler V, Litvinov R I, Lozhkin A P, Peshkova A D, Lebedeva T, Ataullakhanov F I, Spiller K L, Cines D B, Weisel J W. Kinetics and mechanics of clot contraction are governed by the molecular and cellular composition of the blood. Blood. 2016 Jan. 7; 127(1):149-59. doi: 10.1182/blood-2015-05-647560. Epub 2015 Nov. 24. MID: 26603837; PubMed Central PMCID: PMC4705605.
26 Simmons J W, Powell M F. Acute traumatic coagulopathy: pathophysiology and resuscitation. Br J Anaesth. 2016 December; 117(suppl 3):iii31-iii43. Review. Erratum in: Br J Anaesth. 2017 Dec. 1; 119(6):1245. PubMed PMID: 27940454
27 Mikaelsson M. E. (1991) The Role of Calcium in Coagulation and Anticoagulation. In: Sibinga C. T. S., Das P. C., Mannucci P. M. (eds) Coagulation and Blood Transfusion. Developments in Hematology and Immunology, vol 26, Springer, Boston, Mass.

The relevant teachings of all patents, patent applications and references cited herein are incorporated by reference in their entirety.

What is claimed is:

1. A method of forming a therapeutic hemoglobin-based oxygen carrier solution, comprising directly combining at least one of freeze-dried platelets and freeze-dried plasma with a hemoglobin-based oxygen carrier to form a therapeutic hemoglobin-based oxygen carrier solution.

2. The method of claim 1, wherein the hemoglobin-based oxygen carrier includes polymerized hemoglobin.

3. The method of claim 2, wherein the hemoglobin-based oxygen carrier includes a concentration of calcium chloride of greater than 0.03 mMol/L, a concentration of N-acetyl cysteine of greater than 0.31 mMol/L, a concentration of sodium chloride of less than 76 mMol/L, a concentration of potassium chloride of less than 2.7 mMol/L, a sodium hydroxide concentration of less than 8.3 mMol/L, and a concentration of sodium lactate of less than 18.1 mMol/L.

4. The method of claim 2, wherein the hemoglobin is polymerized by reaction with at least one dialdehyde selected from glutaraldehyde, glycolaldehyde, malondialdehyde, succinaldehyde, adipaldehyde, heptanedial, octanedial or 1,9-Nonanedione (2-9 carbon dialdehyde).

5. (canceled)

6. The method of claim 2, wherein the hemoglobin is derived from at least one source from a group consisting of bovine, porcine, human and Arenicola marina (sea worm).

7. (canceled)

8. The method of claim 2, wherein the polymerized hemoglobin is a purified, filtered stroma-free hemoglobin-based oxygen carrier solution of heat-treated bovine hemoglobin that has an average molecular weight ranging from about 130-500 kD MW.

9. The method of claim 8, wherein the hemoglobin-based oxygen carrier includes glutaraldehyde-stabilized and polymerized bovine hemoglobin, sodium chloride, sodium hydroxide, potassium chloride, calcium chloride, sodium lactate and N-acetyl cysteine.

10. The method of claim 8, wherein the hemoglobin-based oxygen carrier includes glutaraldehyde-stabilized and polymerized bovine hemoglobin, calcium chloride and N-acetyl cysteine, in which sodium chloride, sodium hydroxide, potassium chloride and sodium lactate are present at subphysiological concentrations or are absent.

11. The method of claim 8, wherein the hemoglobin-based oxygen carrier includes glutaraldehyde-stabilized and polymerized bovine hemoglobin, and calcium chloride, in which sodium chloride, sodium hydroxide, potassium chloride and sodium lactate exist at subphysiological concentrations or are absent and N-acetyl cysteine is at subphysiological concentrations of cysteine or is absent.

12. The method of claim 8, wherein the hemoglobin-based oxygen carrier is directly combined with freeze-dried platelets.

13. The method of claim 8, wherein the hemoglobin-based oxygen carrier is directly combined with freeze-dried plasma.

14. The method of claim 8, wherein the hemoglobin-based oxygen carrier is directly combined with both freeze-dried platelets and freeze-dried plasma in a 1:1:1 ratio by volume.

15. (canceled)

16. The method of claim 8, wherein the volume ratio of hemoglobin-based oxygen carrier to at least one of freeze-dried platelets and freeze-dried plasma is in a range of 1:3 to 5:1.

17. The method of claim 8, wherein the freeze-dried platelets and freeze-dried plasma are combined by admixing these freeze-dried products prior to rehydration or by rehydrating either of these freeze-dried products with the HBOC-rehydrated formulation of the other freeze-dried product in either order.

18. A therapeutic hemoglobin-based oxygen carrier solution formed by a method of claim 1.

19.-34. (canceled)

35. A method of treating bleeding or anemia and simultaneously enhancing systemic convective oxygen delivery in a subject suffering low circulatory oxygen transport or bleeding, comprising administering to the subject in need thereof a therapeutically effective amount of a therapeutic hemoglobin-based oxygen carrier solution formed by directly combining at least one of freeze-dried platelets and freeze-dried plasma with a hemoglobin-based oxygen carrier.

36.-48. (canceled)

49. The method of treatment of claim 35, wherein the subject is in need of the treatment as a consequence of at least one member of the group consisting of ischemia, hypoxia, and acute bleeding.

50. The method of treatment of claim 35, wherein the ischemia or hypoxia is due to at least one member of the group consisting of bleeding, circulatory hypovolemia, anemia, poor cardiac function, poor pulmonary function, vasoconstriction and vascular occlusion, where vascular occlusion is due to vascular disease or thrombosis; or wherein the bleeding is due to at least one member of the group consisting of blunt or penetrating trauma, depletion of platelets, depletion of coagulation factors, dilution of platelets, dilution of coagulation factors, bone marrow disease, liver injury, and liver disease; or wherein the depletion of at least one of platelets and coagulation factors is due to consumption of these blood components in the subject due to injury or tissue damage.

51.-54. (canceled)

55. A method of forming a therapeutic hemoglobin-based oxygen carrier solution comprising reconstituted platelets, plasma or combination of reconstituted platelets and plasma, comprising reconstituting at least one of dried platelets and dried plasma directly with a hemoglobin-based oxygen carrier to form a therapeutic hemoglobin-based oxygen carrier solution comprising reconstituted platelets, plasma or combination of reconstituted platelets and plasma.

56. A method of treating bleeding or anemia and simultaneously enhancing systemic convective oxygen delivery in a subject suffering low circulatory oxygen transport or bleeding, comprising:
performing the method of claim 55 to produce a therapeutic hemoglobin-based oxygen carrier solution comprising reconstituted platelets, plasma or combination of reconstituted platelets and plasma; and
administering to the subject in need thereof a therapeutically effective amount of the therapeutic hemoglobin-based oxygen carrier solution comprising reconstituted platelets, plasma or combination of reconstituted platelets and plasma.
